(19) United States
(12) Patent Application Publication
(10) Pub. No.: US 2024/0331825 A1
(43) Pub. Date: Oct. 3, 2024

(54) SYSTEM AND METHOD FOR GENERATING ADAPTIVE SUGGESTIONS FOR PATIENT CARE PLANS

(71) Applicant: KONINKLIJKE PHILIPS N.V., EINDHOVEN (NL)

(72) Inventors: ERAN SIMHON, BOSTON, MA (US); LASITH ADHIKARI, REVERE, MA (US); DAVID PAUL NOREN, SHARON, MA (US); GREGORY BOVERMAN, READING, MA (US); CHAITANYA KULKARNI, BANGALORE (IN); SYAMANTHAKA BALAKRISHNAN, BANGALORE (IN); VIKRAM SHIVANNA, BANGALORE (IN); LARRY JAMES ESHELMAN, OSSINING, NY (US)

(21) Appl. No.: 18/576,394

(22) PCT Filed: Jun. 27, 2022
(86) PCT No.: PCT/EP2022/067595

§ 371 Date: Jan. 4, 2024

Related U.S. Application Data
(60) Provisional application No. 63/218,517, filed on Jul. 6, 2021.

(51) Int. Cl. G16H 20/00; G16H 50/70

(52) U.S. Cl. CPC G16H 20/00; G16H 50/70

(57) ABSTRACT

Generally, in one aspect a process for providing adaptive suggestions in the CPOE is provided. The process includes: (i) generating a rule set from historic patient data (ii) receiving data specific to a patient (iii) determining, by a processor of the adaptive suggestion system containing a set of created rules, the most likely entry for that component of the CPOE based on the rule set and received patient data (iv) displaying via user interface the suggested entry for the component of the CPOE.

### FIELD OF THE DISCLOSURE

The present disclosure is directed generally to methods and systems for generating adaptive suggestions for patient care plans.

### BACKGROUND

Digital record systems such as Electronic Medical Records (EMR) are used in modern health care to manage patient records. Creating and updating Computerized Physician Orders Entries (CPOEs) is one of the main EMR related activities done by physicians or other medical professionals. Typically, a CPOE is created upon a patient's admission and updated throughout the patient's stay (for example, when there is a change in the dosage of a given medication). CPOEs have many components to fill out and should include the complete care plan of a patient including lab tests, medications, nutrition, etc. At present, the act of filling out a CPOE can require hundreds of clicks and entering each piece of information manually by text, drop down list, or combo box. FIG. 1 is a screenshot from the Philips TASY CPOE module. As shown, there are numerous entry fields to complete on the Infusion and Medication portion including item, route, dose, unit of measurement, interval, start, and end.

Many physicians are facing EMR fatigue from the burdensome and time-consuming process of completing CPOEs and documenting patient records. Research shows that on average physicians spend two hours in front of the computer for every hour they spend with patients. This results in physicians having less time for patient interaction and many physician's experiencing burnout. Reducing EMR burden is a key component for operations excellence in hospitals and is aligned with both provider satisfaction and safety, two axes of the quadruple aim.

While the use of artificial intelligence in EMR systems has had a pronounced impact on provider satisfaction and efficiency the process is still extremely time consuming. Some conventional digital medical record systems have implemented decision support tools to increase safety, for example by triggering alerts and including information for items such as dosage or adverse effects. Other systems and devices have implemented methods to refine the use of decision support tools to reduce gaps and inconsistencies in patient records. However, these systems do not reduce the time it takes a physician to complete a CPOE or streamline their workflow. Accordingly, there is a need for systems and methods to reduce EMR burnout by cutting down the time it takes physician's to complete CPOEs.

### SUMMARY OF THE DISCLOSURE

There is a continued need for methods and systems to streamline and improve the order entry process for CPOEs to reduce EMR fatigue. The present disclosure is directed at inventive methods and systems for generating suggestions for EMR components using a machine learned algorithm based on historic data. Providing suggestions for EMR components informs the physician of likely paths forward rather than triggering alerts or blockages which consume more time. This results in physicians spending less time creating care plans and less physicians experiencing EMR burnout.

Various embodiments and implementations herein are directed to systems and methods for providing physicians with adaptive suggestions for completing CPOEs based on a statistical analysis of data. The system uses information from records relevant to care plans to create association rules. These association rules can be used to provide suggested entries for physicians while they are completing care plans using a CPOE system. The adaptive suggestion system can display the suggestions and statistical information on the user interface so that the physician can choose to accept or decline the suggestions. As the physician enters more information into the CPOE the system is able to provide better suggestions for the remaining components. The system can also streamline the CPOE process by anticipating the next component.

Generally, in one aspect a process for providing adaptive suggestions in the CPOE is provided. The process includes: (i) receiving data specific to a patient (ii) receiving a trigger for a specific entry field by a physician selecting a component of a CPOE to fill out for that patient (iii) determining, by a processor of the adaptive suggestion system containing a set of created rules, the most likely entry for that component of the CPOE based on the rules and received patient data (iv) displaying via user interface the suggested entry for the component of the CPOE with statistical information (v) suggesting additional changes in the current care plan given a change in the care plan or in other relevant data point.

According to one embodiment, the process includes the steps of generating, by a processor of the adaptive suggestion system, a rule set for obtaining suggested entries for each of a plurality of components of the CPOE; receiving, by the adaptive suggestion system, information specific to the patient; determining, by the processor of the adaptive suggestion system using and algorithm to apply the generated rules to the received information specific to the patient, (1) a suggested entry for the component of the CPOE (2) a support metric for the suggested entry (3) a confidence metric for the suggested entry; and displaying the suggested entry for the component of the CPOE on a display of the adaptive suggestion system.

According to an aspect, the process further comprises the steps of: receiving, by the adaptive suggestion system a dataset, the dataset comprising past care plans for each of a plurality of patients and extracting and normalizing the received data.

According to an aspect, the process further comprises the step of displaying the determined confidence metric for the determined suggested entry alongside the displayed suggested entry.

According to an aspect, the process further comprises the step of displaying the determined support metric for the determined suggested entry alongside the displayed suggested entry.

According to an aspect, the rule set is comprised of a plurality of rules each having a left hand side and a right hand side wherein the left hand side corresponds to information specific to the patient and the right hand side corresponds to a possible suggested entry for the component of the CPOE.

According to an aspect, the process further comprises the steps of compiling a list of all possible rules from the rule set that could be applied to the information specific to the patient; and determining a confidence metric for each of the possible rules.

According to an aspect, the process further comprises the step of removing rules from the rule set that do not have a determined confidence metrics that meets a pre-determined threshold.

In another proposed embodiment the process comprises following main steps: generating, by a processor of the adaptive suggestion system, a rule set for obtaining suggested entries for each of a plurality of components of the CPOE; receiving, by the adaptive suggestion system, information specific to the patient; determining, by the processor of the adaptive suggestion system using an algorithm to apply the generated rule set to the received information specific to the patient, (1) a suggested entry for a first component of the CPOE (2) a support metric for the suggested entry for the first component of the CPOE (3) a confidence metric for the suggested entry for the first component of the CPOE; displaying the suggested entry for the first component of the CPOE on a user display of the adaptive suggestion system; completing (170) the entry for the first component of the CPOE; determining, by the processor of the adaptive suggestion system using an algorithm to apply the generated rule set to the received information and the completed entry for the first component of the CPOE, (1) a suggested entry for a second component of the CPOE (2) a support metric for the suggested entry for the second component of the CPOE (3) a confidence metric for the suggested entry for the second component of the CPOE; and displaying the suggested entry for the second component of the CPOE on the user display of the adaptive suggestion system.

According to an aspect, the process further comprises the steps of: receiving, by the adaptive suggestion system a dataset, the dataset comprising past care plans for each of a plurality of patients; and extracting and normalizing the received data.

According to an aspect, the process further comprises the step of displaying the determined confidence metric for the determined suggested entry alongside the displayed suggested entry.

According to an aspect, the process further comprises the step of displaying the determined support metric for the determined suggested entry alongside the displayed suggested entry.

According to an aspect, the rule set is comprised of a plurality of rules each having a left hand side and a right hand side wherein the left hand side corresponds to information specific to the patient and the right hand side corresponds to a possible suggested entry for the component of the CPOE.

According to an aspect, the process further comprises the steps of compiling a list of all possible rules from the rule set that could be applied to the information specific to the patient and determining a confidence metric for each of the possible rules.

According to an embodiment the adaptive suggestion system comprises a set of rules corresponding to relationships obtained from medical information from a plurality of patients; an algorithm configured to generate a suggestion for each of a plurality of components for the CPOE using the set of rules; a processor configured to: determine, using the rule set, the algorithm, and an item list comprising information received pertaining to the patient, (1) a suggested entry to a CPOE component, (2) a support metric for the suggested entry (3) a confidence metric for the suggested entry; and a display configured to display the determined suggested entry.

According to an aspect the system further comprises user interface, wherein the user interface is configured to receive an entry for the component of the CPOE.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a prior art screenshot of the Philips TASY CPOE module.

FIG. 2 is a flowchart of a method for providing adaptive suggestions in the CPOE using the adaptive suggestion system, in accordance with an embodiment.

FIG. 3 is a graphic user interface of a portion of the adaptive suggestion system, in accordance with an embodiment.

FIG. 4 is a graphic user interface of a portion of the adaptive suggestion system, in accordance with an embodiment.

FIG. 5 flowchart of a method for providing adaptive suggestions in the CPOE using the adaptive suggestion system, in accordance with an embodiment.

FIG. 6 is a schematic representation of an adaptive suggestion system, in accordance with an embodiment.

### DETAILED DESCRIPTION OF EMBODIMENTS

The present disclosure describes various embodiments of a system and method configured to generate suggested entries for components of a patient care plan. Applicant has recognized and appreciated that it would be beneficial to provide a method and system to generate suggestions for CPOE components using a machine learned algorithm based on a statistical analysis of historic data. Applicant has further recognized and appreciated that it would be beneficial to provide a method and system that can provide suggested updates to care plans. The system and method disclosed will help to make the patient care plan process more efficient by creating rules based on historic input data and leveraging patient information and the previously completed care plan components to suggest other care plan components.

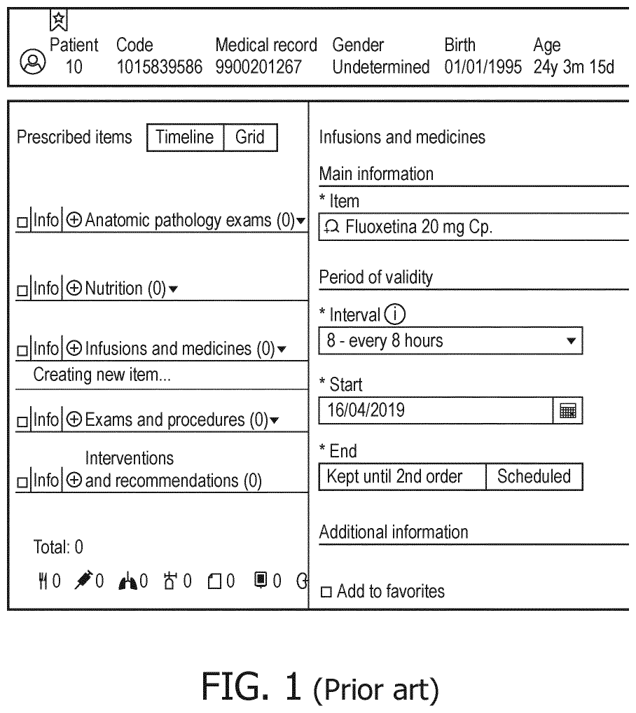

A patient care plan is used by providers to enter and send treatment instructions for a patient—including medication, laboratory, nutrition, and radiology orders. A CPOE is an electronic method of entering these orders. A CPOE is made up of multiple components. Referring now to FIG. 1, shown is a screenshot of an example of a portion of a conventional CPOE system, for ordering infusions and medicines. This is one portion of the CPOE and is comprised of multiple components including item, route, dose, unit of measurement, and more. A physician would be required to enter a response for each component. According to one embodiment, the adaptive suggestion system provides a system and method to predictively suggest responses for each component of the CPOE while a physician is in the process of filling out an individual's CPOE.

As discussed in greater detail herein, the adaptive suggestion system 200 uses data from records such as previous care plans to generate rules based on associations found in the data. While a physician is entering a CPOE for a patient, the system uses the generated rules and information specific to the patient to generate and display suggested entries for the components of the CPOE. The physician can either accept or reject each components suggestion. The system will use each new entry to provide better suggestions for the remaining components. The system can also streamline the process by optimizing the order the components are presented to the physician in.

While the disclosure focuses on adaptive suggestions for completing CPOEs it should be known that the system and method disclosed can be applied to any other suitable electronic documents, records, forms, and processes. The system and method can be a standalone system, or it can be integrated into current EMR and CPOE systems such as Philips TASY EMR solution.

The adaptive suggestion system 200 may be embodied in whole or in part within a device. For example, the entire system may be embodied within a single device such as a handheld device, laptop, computer, or other single device. Alternatively, the adaptive suggestion system 200 may comprise a user interface that is transportable or remote, such as a handheld device, mobile phone application, computer, or other transportable element that functions as a user interface and/or communication interface. The device will communicate the information to another remote or transportable component or network portion of the system for analysis. The results of the system may then be communicated back to the transportable user interface or stored in another portion of the system 200.

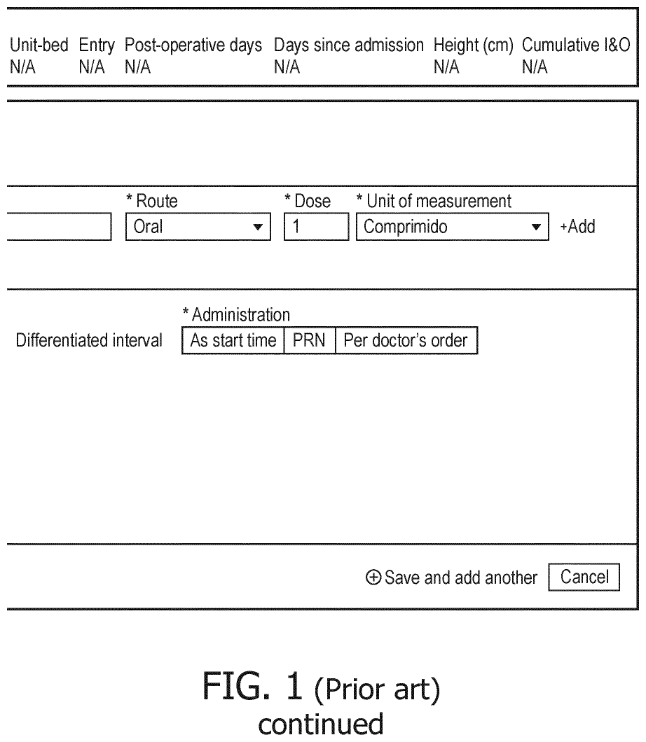

Referring to FIG. 2, a flowchart of one embodiment of a method 100 for generating suggestions for CPOE components, using the adaptive suggestion system 200 is depicted. The methods and systems described in connection with the figures are provided as examples only, and shall be understood not to limit the scope of the disclosure. The adaptive suggestion system 200 can be any of the systems described or otherwise envisioned herein. The adaptive suggestion system can be a single system or multiple different systems.

At step 110 of the method 100, the system receives historical medical data for which associations and rules will be made. According to one embodiment this data includes previous CPOEs or other patient care plans. This information can also include other records that can be used to correlate relationships between patient data and care plan components. The data can include patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. Additionally, this information can include any component of a care plan such as medication given, procedures, lab tests, exams and more. Many other types of medical information are possible as well.

The data received in this step may be stored in and/or received from one or more databases. The database may be a local and/or remote database. For example, the adaptive suggestion system 200 may comprise a dataset of historical patient data or the system may be in communication with a database such as a local or remote EMR database. The database can also be received from publicly available datasets, for example MIMIC-III which is a freely accessible critical care database. The database may be a private dataset, such as one or in connection with the facility using the system. According to an embodiment, the system comprises an EMR database or system which is optionally in direct and/or indirect communication with system 200.

At step 120 of the method 100, the adaptive suggestion system 200 extracts and normalizes the data received in step 110. At this step, the system can extract features specific to patient care plans from the received data and normalizes the data to be able to identify associations between the features. This can be accomplished by a variety of embodiments for feature identification, extraction, normalization and/or processing, including any method or algorithm for extracting and normalizing data.

According to an embodiment, the system may comprise a data pre-processor or similar component or algorithm configured to process the received data. For example, the data pre-processor analyzes the data to remove noise, bias, errors, and other potential issues. The data pre-processor may also analyze the input data to remove low quality data. Many other forms of data pre-processing or data point identification and/or extraction are possible. The system in this step the system can also group data into categories such as but not limited to age ranges.

At step 130, using the extracted normalized data the system 200 generates a rule set. The rule set can then be stored in either the memory, storage, or other storage device. The rule set can be used later to generate suggested care plan component data entries based on associations among features.

According to one embodiment, the generated rules are association rules. Association rules can correlate the frequency of co-occurrences in the received data. Association in this context can, refer to any correlation between features, not just those that predict a particular class or value. This step can also include analyzing the data to find patterns, perform feature evaluation, perform feature subset selection, develop predictive models, and understand interactions between features.

The rule set can be generated using any machine learning algorithm trained with the received historical data. According to one embodiment, association rules can be created efficiently using an existing tool like the APRIORI algorithm which identifies the frequent individual items in the database and extends them to larger and larger item sets as long as those item sets appear sufficiently often in the database. The frequent item sets determined by the APRIORI algorithm can be used to determine the rule set. Alternatively, any other association rule-based algorithm or method as should be known in the art can be used such as but not limited to Eclat or FP-growth algorithms.

In another embodiment, the purpose of the rule set can be accomplished using a machine trained classifier that will be used to generate suggestion responses. The machine trained classifier can be trained using the data received in step 110. A classifier can be generated based on any known machine learning methods or models such as but not limited to linear regression, logistic regression, decision trees, support vector machine, naive bayes, k-nearest neighbors, k-means, random forest, dimensionality reduction algorithms, or gradient boosting algorithms including GBM, XGBoost, LightGBM, and CatBoost.

According to one embodiment, rules are created for each possible component of a patient care plan. For example, the rule set includes rules associated with every possible order such as but not limited to every medication, lab test, exam, therapy, and nutrition order.

According to one embodiment, each rule comprises a Left-Hand Side (“LHS”) and Right-Hand Side (“RHS”). For example, associated rules can be denoted by:

{A,B}→{C}. (Eq. 1)

In this example the LHS of the rule is A, B and the RHS is C. This rule would signify that if A and B are present then C is also present. The strength of a rule can be measured by two metrics: support and confidence. In other embodiments lift is also measured. Support refers to how often a given rule appears in the received data. Confidence refers to the percentage value that shows how frequently the given rule is true in the received data.

The system calculates support as the number of instances that follow the rule (i.e., includes all items from LHS and RHS). In the example, if there are 500 instances in the received data where C is present when both A and B are present, meaning A, B, and C are all present in one care plan, the support is equal to 500. The support equation for this example is shown below:

Support({A,B}→{C})=500 (Eq. 2)

The system calculates confidence as the ratio between the number of instances that include all items from both the LHS and the RHS and the number of instances that include the LHS. In the example where there are 500 instances of C being present when both A and B are present confidence can be measured by calculating the total number of instances with A, B, or C and then dividing that number by the calculated 500. An example where 80% of the care plans from the received data that included items A, B, or C included all three A, B, and C is illustrated below:

Confidence({A,B}→{C})=0.8 (Eq. 3)

According to an embodiment, during this step, all possible rules and the support and confidence level of each rule is generated and stored. According to another embodiment, the generated rule set can be filtered by a threshold. For example, to avoid having a huge number of rules, the algorithm can store only rules with confidence and/or support higher than a pre-defined threshold.

The generated rule set can be static, or dynamic such that it is updated or re-calculated using subsequently available data. Updating or recalculating the rules can be constant or periodic. According to one embodiment, the rules are regenerated weekly and at each update, data from care plans of the recent week will be added. In another embodiment, the oldest data, for example from the first week of data can be removed prior to regenerating rules in order to guarantee that new clinical protocols and medications will be considered.

According to one embodiment, the system 200 uses a user interface and/or communication interface to allow the user to manually create rules. Manual rules can be added when a new treatment or medication is introduced, and there is still not enough historic data for the rule to be created automatically. Manual rules may also be beneficial to reflect medical recalls, policy changes, or preferences of the physician, hospital, or network.

At step 140, for each care plan the system 200 receives data specific to the patient who the care plan is for. When a physician initiates a new care plan basic information about the patient/encounter can be extracted or entered. The adaptive suggestion system 200 may obtain the patient's information manually or automatically. In one embodiment the system extracts patient information from the EMR database, monitored vitals, and receives any missing data by manual entry using the communication or user interface.

The patient information received in this step may be used by the system in the next step to generate the item list or the LHS of the algorithm. Patient information can be stored in either the memory, storage, or other storage device of the system. This can be done when information about the patient can be received from a database or manually entered into the system by the user.

At step 150, the system 200 can determine, by a processor of the adaptive suggestion system 200 containing an algorithm, the set of generated rules, and the patient specific data, a suggested entry for a component of the CPOE. In this step the system can generate a list of rules that have a LHS matching the patient specific data then determine the best rule to use.

This step can be triggered by a user entering, editing, or starting a CPOE for a patient, or by the user opening a specific component or portion of the CPOE. According to an embodiment, at this step the system 200 will determine a suggested entry for a triggered component of the CPOE. Alternatively, the system can suggest a response to a component of the CPOE in a predetermined order as discussed later in step 180.

The patient information received in step 140, comprises the item list that will be used to correspond to the LHS of the rules. For example, if the care plan is being completed for an 82-year-old with diabetes who is being seen for chest pain, the system would receive this information in step 140. An example of the item list for this patient is:

Item List={age=82,reason for visit=“chest pain”,comorbidity=diabetes} (Eq. 4)

According to an embodiment, the system 200 can use the algorithm to find all rules from the generated rule set that have an LHS equal to the item list. The system 200 can use the algorithm to create permutations of the item list that do not include all items and find the rules corresponding to each permutation.

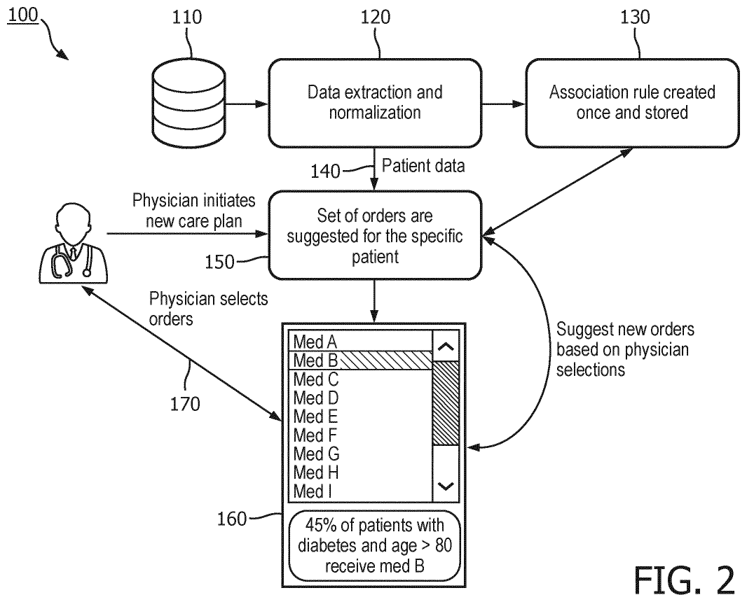

In one embodiment, the system can generate a pop up or window on the user interface to ask the physician if there are specific items that should be removed from the item list, an example of which is depicted in FIG. 3. This may be used to remove any outlying or non-relevant patient data and to improve the accuracy of the suggestions.

For each empty component of the CPOE, the algorithm can determine a suggested entry that corresponds to the RHS column of the rules. Using the rules generated from the received data and the item list the algorithm will compile the list of possible rules. This list will include all rules from the rule set that have a LHS that includes items from the item list. Table 1 below illustrates the set of rules for the medicine component of the CPOE for the patient with an item list of age=82, reason for visit=“chest pain”, comorbidity=diabetes. As shown, the LHS column has different permutations of the item lists.

TABLE 1

| LHS | RHS | Support | Confidence |
|---|---|---|---|
| age > 80, reason for visit = “chest pain” | Med A | 1000 | 0.8 |
| reason for visit = “chest pain”, | Med A | 800 | 0.75 |
| comorbidity = deiabetes | | | |
| age > 08, reason for visit = | Med A | 500 | 0.5 |
| “chest pain”, comorbidity = deiabetes | | | |
| age > 80, comorbidity = deiabetes | Med B | 2000 | 0.4 |
| comorbidity = deiabetes | Med C | 300 | 0.3 |
| age > 80 | Med C | 2500 | 0.2 |
| age > 80, reason for visit = “chest pain” | Med D | 1000 | 0.3 |

The support column can be used as a threshold for removing rules with low support (i.e., ignore rules with less than 100 instances). In the example shown in Table 1, the complied rules indicate Med A is present in 1000 care plans from the received data where the age of the patient is over 80 and the patient's reason for visit is “chest pain.” If a support threshold was set at 100 instances this rule would still be compiled as it is above 100. The confidence column can be used to sort rules and to recommend the rules/items with the highest confidence. Here, the rule for Med A where age is over 80 and the patient's reason for visit is “chest pain” has a confidence of 0.8, which is the highest confidence from the complied list.

According to one embodiment, the system 200 can determine the suggested entry for the component of the CPOE is equal to the RHS of the rule with the highest confidence from the list of rules found to correspond to the item list or permutations of the item list. In the example shown in table 1, the system would determine the suggested entry would be Med A for the medicine component of this CPOE. Alternatively, the system can determine to suggest all medications found in the RHS of the rules that have an LHS matching the item lists or permutations of the item list. The determined suggestions can be a list of all rules sorted by their confidence.

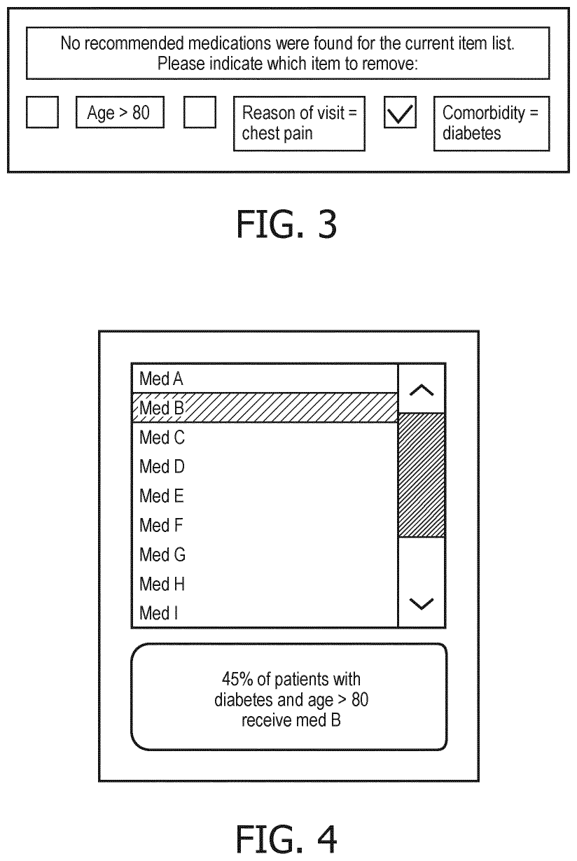

At step 160, the system displays the suggested entry for the component to the user on the user interface. An example of the suggestion being displayed is depicted in FIG. 4.

According to an embodiment, if there are multiple suggestions for the same component then the rule with the highest confidence will be displayed. Alternatively, the system can use the user interface to present to the user a list of suggestions with statistical information that explains why these items are suggested. For example, each suggested entry for the component of the CPOE and the support and confidence can be displayed. The outcome can be a sorted list/combo box, where each item is a suggestion sorted by the confidence of the rule.

According to another embodiment, the user can start typing an entry for a component using the communication interface and the system can auto complete the entry based on the determined suggestions.

In step 170, the user can choose to approve the suggested entry or to deny the suggested entry and input a different entry. The entry for this component of the CPOE is complete, is then used to improve future component suggestions. The algorithm iteratively suggests new order items based on existing items of the care plan. In part, because changing one component of the plan can influence other components. For example, prescribing new medication, might influence diet instructions. Further because the entry for this item is part of the item list for demining suggestions for other components.

Once a suggestion is accepted by the physician, then the algorithm is recomputed where the accepted component will become of the LHS and will no longer be considered for the RHS. In the example from Table 1, once the user completes the medications component based on the suggestion or by manually entering medications, a new table will be created for the next component in the care plan, and the medication orders will be part of the LHS. In the example illustrated in Table 1, if the physician chose the suggested Med A the system would then consider Med A part of the item list when generating a suggestion for the next component. Table 2 illustrates this where the system is generating rules for labs. Med A is part of the LHS and labs are shown for RHS.

TABLE 2

| | | Sup- | Confi- |
|---|---|---|---|
| LHS | RHS | port | dence |
| age = 82, reason for visit = “chest pain”, | lab A | 400 | 0.7 |
| med = A | | | |
| reason for visit = “chest pain”, comorbidity = | lab A | 300 | 0.65 |
| deiabetes | | | |
| age = 82, reason for visit = “chest pain”, | lab A | 400 | 0.5 |
| comorbidity = deiabetes, med = A | | | |
| age = 82, comorbidity = deiabetes, med = A | lab B | 1200 | 0.4 |
| comorbidity = deiabetes | lab C | 400 | 0.3 |
| age = 82, med = A | lab C | 2000 | 0.2 |
| age = 82, reason for visit = “chest pain” | lab C | 700 | 0.4 |

What follows is pseudo-code example of the above-described algorithm step of the method:
1. List of components-{meds, labs, nutrition . . . }
2. Initiate patient's items list
3. For component in list of components
4. Create empty list of rules
5. For each item in component
6. For each permutation of Items list
7. Find association rule and store in list or rules
8. Present to the user the rule with the highest confidence
9. If item in the presented rule is accepted, then add item to Item list and go to Step 4
10. If rejected, remove item and go to Step 6
11. If user click “move to next component” go to Step 2

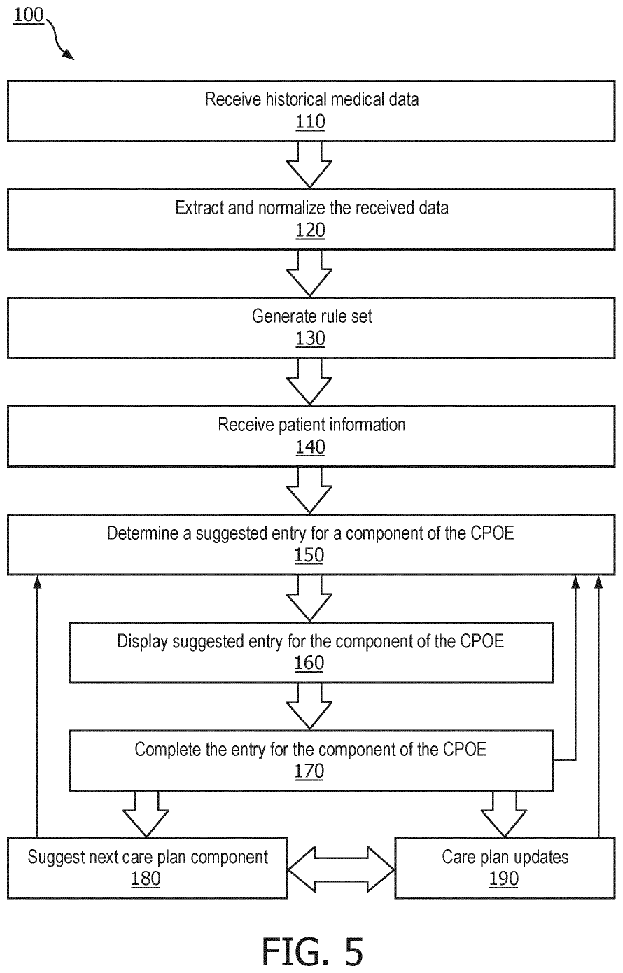

Referring now to FIG. 5, the process 100 can also include steps 180 and 190. In step 180, the system 200 can optimize the order that the components of the care plan are presented in. In this step the system 200 can dynamically sort the list of components that are included in the CPOE system (labs, medications, diet . . . ) to present the components in an order that would decrease the total overall manual data entering.

According to one embodiment, the system 200 can begin by providing care plan suggestions in a randomized order of components. For example, one user can get suggestions for medications first, then lab tests, and so on. While other user first gets lab test suggestions and then medication suggestions. As the user's complete the CPOEs the system 200 can record statistical data such as but not limited to acceptance rate of suggest items, components skipped, and total time spent completing the CPOE. Once enough statistical data is collected, the system can identify which order of care plan components is optimal.

In step 190, the system 200 can update suggestions for care plan components. According to an embodiment new suggestions can be generated at periodic increments or when a change or update has occurred. This can be done at any time and during or after the stay. For example, the system can retrigger steps 150 and 160 after an event such as but not limited to a change in the care plan, a new manual rule set in place, a new rule set is generated, or at discharge. Alternatively, the system can retrigger steps 150 and 160 at periodic increments.

During this step, all items of the completed plan will be part of the item list, and the algorithm can compile all possible rules for each component. According to an embodiment, when there is already a complete care plan in place, new suggestions will be made only if the algorithm finds rules with a very high confidence for example over a pre-determined threshold. If no such rules exist, then new suggestions will not be made. A benefit of this aspect is that if an ordered medication has a side effect that impacts most patients and can be mitigated with another medication, then ideally, the algorithm will suggest adding it to the care plan.

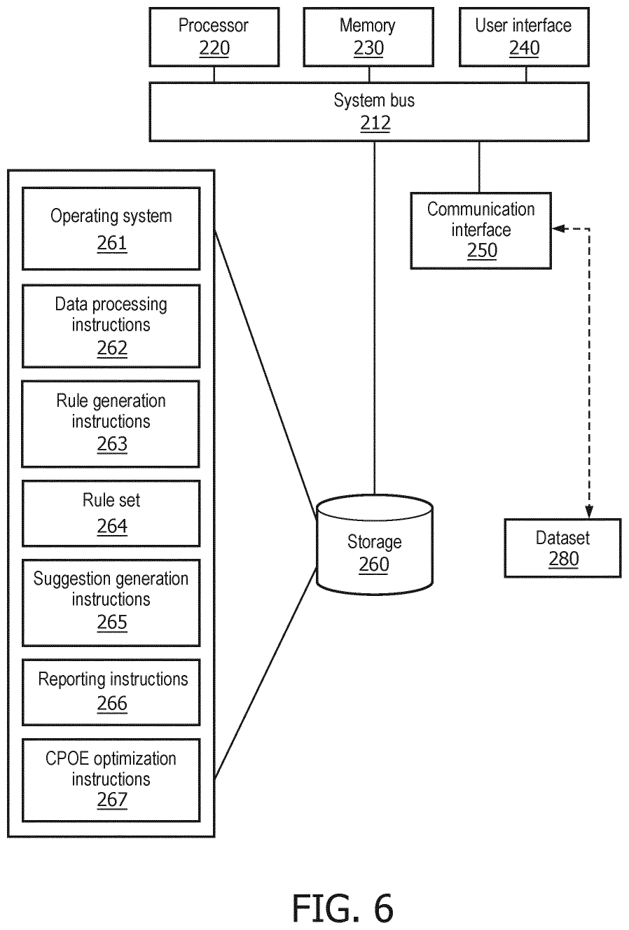

Referring now to FIG. 6, according to an embodiment of system 200, the system comprises one or more of a processor 220, memory 230, user interface 240, communication interface 250, storage 260, interconnected via one or more system buses 212. It will be understood the system constitutes, in some respects, an abstraction and that the actual organization of the components of the system 200 may be different and more complex than illustrated. Additionally, system can be any of the systems described or otherwise envisioned herein. Other elements and components of system are disclosed and/or envisioned elsewhere herein.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, system 200 may comprise or be in remote or local communication with a database, data set, or data source 280. Dataset 280 may be a single database or data source or multiple. Dataset 280 may comprise the input data which may be used to generate the rule set or train the classifier, as described and/or envisioned herein.

According to an embodiment, storage 260 of system 200 may store one or more algorithms and/or instructions to carry out one or more functions or steps of the methods described or otherwise envisioned herein. For example, processor 220 may comprise one or more of data processing instructions 262, rule generation instructions 263, rule set 264, suggestion generation instructions 265, reporting instructions 266, and/or CPOE optimization instructions 267.

According to an embodiment, data processing instructions 262 direct the system to retrieve and process input data which is used to either: (i) create the rule set using the rule generation instructions 263, or (ii) to process the patient specific data for use in generating suggestions. The data processing instructions 262 direct the system to, for example, receive or retrieve input data or medical data to be used by the system as needed, such as from database 215 among many other possible sources. As described above, the input data can comprise a wide variety of input types from a wide variety of sources.

According to an embodiment, the data processing instructions 262 also direct the system to process the input data to extract and normalize the data related to care plans for a plurality of patients, which are used generate the rule set. This can be accomplished by a variety of embodiments for feature identification, extraction, and/or processing. The outcome of the data processing is a set of normalized features related to patient care plans.

According to an embodiment, rule generation instructions 263 direct the system to utilize the processed data to generate the rule set. Thus, the system comprises a generated rule set 264 configured to determine a suggested entry for the CPOE. These instructions can also include when to regenerate rules and manually entered rule procedures.

The rule set 264 is configured to determine a suggested entry for the CPOE using the individual patient's data using the suggestion generation instructions 265. The suggestion generation instructions 265 are configured to use the received data and the rule set 264 to generate the suggested entry. The suggestion can comprise a suggested treatment or test, for example, a medicine, a lab, an exam, a diet, or other. Preferably, the adaptive suggestion system and machine learned rule set educate the entering physician and decrease the time it takes to complete a CPOE.

According to an embodiment, reporting instructions 266 direct the system to generate and provide the suggestion and in some example any addition information such as confidence and support. The suggestion may comprise many different configurations and information. For example, the suggestion may only provide the top result or it could provide all results, or results over a certain threshold. In some cases, there may be no suggestion provided. The suggestion may further comprise an indication of which of support and confidence. The reporting instructions 266 also direct the system to display the suggestion on a display of the system. The many different configurations and information. For example, the suggestion may only provide the top result or it could provide all results, or results over a certain threshold. In some cases, there may be no suggestion provided. The suggestion may further comprise an indication of which of support and confidence. Other information is possible such as but not limited to information regarding dosage, interactions, or warnings. Alternatively, the suggestion may be communicated by wired and/or wireless communication to another device. For example, the system may communicate the suggestion to a mobile phone, computer, laptop, wearable device, and/or any other device configured to allow display and/or other communication of the suggestion.

According to an embodiment, the CPOE optimization instructions 267 are configured to determine when the previously entered orders need to be recalculated or a new suggestion is better. Further the CPOE optimization instructions 267 can be configured to allow the system to suggest the next component for filling out.

According to an embodiment, the adaptive suggestion system is configured to process many thousands or millions of datapoints in the input data used to generate the rule set. For example, generating a functional and skilled rule set or trained classifier using an automated process such as data identification, extraction, and normalization and subsequent rule formation requires processing of millions of datapoints from input data and the generated relationships. This can require millions or billions of calculations to generate a rule set from those millions of datapoints and millions or billions of calculations. As a result, each rule is novel and distinct based on the input data and parameters of the machine learning algorithm. Thus, generating a functional and skilled rule set comprises a process with a volume of calculation and analysis that a human brain cannot accomplish in a lifetime, or multiple lifetimes.

Similarly, the adaptive suggestion system can be configured to continually receive and process medical data, and provide periodic or continual updates to the generated rule set via the processors as well as analyzing each set of data specific to each patient for every component. This requires the analysis of thousands or millions of datapoints on a continual basis to optimize the reporting, requiring a volume of calculation and analysis that a human brain cannot accomplish in a lifetime.

By providing recommendations for CPOE entireties, this novel adaptive suggestion system has an enormous positive effect on reducing EMR burden and fatigue compared to prior art systems. As just one example, by providing a system that can generate the most likely response, the system will improve the time it takes to complete the CPOE. The amount of information needed by a human for adequate deduction of rules based on data from the most recent input data, particularly for a large number of care plans, is likely to be incomplete and incorrect. Additionally, it would take more time than humanly possible. Thus, rule generation by humans is likely to be inefficient and flawed compared to the generation performed by the novel systems and methods described or otherwise envisioned herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A process for generating, using an adaptive suggestion system, suggested entries for a component of a patient's Computerized Physician Orders Entries (“CPOE”), the process comprising the steps of:
generating, by a processor of the adaptive suggestion system, a rule set for obtaining suggested entries for each of a plurality of components of the CPOE;
receiving, by the adaptive suggestion system, information specific to the patient;
determining, by the processor of the adaptive suggestion system using an algorithm to apply the generated rule set to the received information specific to the patient, a suggested entry for the component of the CPOE a support metric for the suggested entry a confidence metric for the suggested entry; and
displaying the suggested entry for the component of the CPOE on a display of the adaptive suggestion system.

2. The process of claim 1, further comprising the steps of:
receiving, by the adaptive suggestion system a dataset, the dataset comprising past care plans for each of a plurality of patients; and
extracting and normalizing the received data.

3. The process of claim 1, further comprising the step of displaying the determined confidence metric for the determined suggested entry alongside the displayed suggested entry.

4. The process of claim 1, further comprising the step of displaying the determined support metric for the determined suggested entry alongside the displayed suggested entry.

5. The process of claim 1, wherein the rule set is comprised of a plurality of rules each having a left hand side and a right hand side wherein the left hand side corresponds to the information specific to the patient and the right hand side corresponds to a possible suggested entry for the component of the CPOE.

6. The process of claim 1, further comprising the steps of:
compiling a list of all possible rules from the rule set that could be applied to the information specific to the patient; and
determining a confidence metric for each of the possible rules.

7. The process of claim 1, further comprising the step of removing rules from the rule set that do not have a determined confidence metrics that meets a pre-determined threshold.

8. A process for completing, using an adaptive suggestion system, a patient's Computerized Physician Orders Entries (“CPOE”), the process comprising the steps of:
generating, by a processor of the adaptive suggestion system, a rule set for obtaining suggested entries for each of a plurality of components of the CPOE;
receiving, by the adaptive suggestion system, information specific to the patient;
determining, by the processor of the adaptive suggestion system using an algorithm to apply the generated rule set to the received information specific to the patient, a suggested entry for a first component of the CPOE a support metric for the suggested entry for the first component of the CPOE a confidence metric for the suggested entry for the first component of the CPOE;
displaying the suggested entry for the first component of the CPOE on a user display of the adaptive suggestion system;
completing the entry for the first component of the CPOE;
determining, by the processor of the adaptive suggestion system using an algorithm to apply the generated rule set to the received information and the completed entry for the first component of the CPOE, a suggested entry for a second component of the CPOE a support metric for the suggested entry for the second component of the CPOE a confidence metric for the suggested entry for the second component of the CPOE; and
displaying the suggested entry for the second component of the CPOE on the user display of the adaptive suggestion system.

9. The process of claim 8, further comprising the steps of:
receiving, by the adaptive suggestion system a dataset, the dataset comprising past care plans for each of a plurality of patients; and
extracting and normalizing the received data.

10. The process of claim 8, further comprising the step of displaying the determined confidence metric for the determined suggested entry alongside the displayed suggested entry.

11. The process of claim 8, further comprising the step of displaying the determined support metric for the determined suggested entry alongside the displayed suggested entry.

12. The process of claim 8, wherein the rule set is comprised of a plurality of rules each having a left hand side and a right hand side wherein the left hand side corresponds to the information specific to the patient and the right hand side corresponds to a possible suggested entry for the component of the CPOE.

13. The process of claim 8, further comprising the steps of:
compiling a list of all possible rules from the rule set that could be applied to the information specific to the patient; and
determining a confidence metric for each of the possible rules.

14. An adaptive suggestion system configured to generate suggestions for completing a patient's Computerized Physician Orders Entries (“CPOE”), comprising:
a set of rules corresponding to relationships obtained from medical information from a plurality of patients;
an algorithm configured to generate a suggestion for each of a plurality of components for the CPOE using the set of rules;
a processor configured to: determine, using the rule set, the algorithm, and an item list comprising information received pertaining to the patient, a suggested entry to a CPOE component, a support metric for the suggested entry a confidence metric for the suggested entry; and
a display configured to display the determined suggested entry.

15. The system of claim 14, further comprising a user interface, wherein the user interface is configured to receive an entry for the component of the CPOE.
